(19) United States
(12) Patent Application Publication
(10) Pub. No.: US 2013/0053457 A1
(43) Pub. Date: Feb. 28, 2013

(54) METHOD FOR NAPHTHALENE REMOVAL

(71) Applicants: Isto EILOS, Porvoo (FI); Jukka Koskinen, Espoo (FI); Kyösti Lipiäinen, Porvoo (FI); Marja Tiitta, Porvoo (FI); Sami Toppinen, Helsinki (FI); Heli Vuori, Espoo (FI); Heidi Österholm, Porvoo (FI)

(72) Inventors: Isto EILOS, Porvoo (FI); Jukka Koskinen, Espoo (FI); Kyösti Lipiäinen, Porvoo (FI); Marja Tiitta, Porvoo (FI); Sami Toppinen, Helsinki (FI); Heli Vuori, Espoo (FI); Heidi Österholm, Porvoo (FI)

(73) Assignee: Neste Oil Oyj, Espoo (FI)

(21) Appl. No.: 13/591,953

(22) Filed: Aug. 22, 2012

Related U.S. Application Data
(60) Provisional application No. 61/526,915, filed on Aug. 24, 2011.

(30) Foreign Application Priority Data
Aug. 24, 2011 (EP) 11178592.9

(51) Int. Cl. C01B 3/56; C07C 1/04

(52) U.S. Cl. 518/703; 252/373

(57) ABSTRACT

A method for naphthalene removal from a gas using a solid adsorbent material including benzene for the removal. The removal can be applied to, for example, a crude syngas main stream and/or a carbon dioxide exhaust side stream. The adsorption to the adsorbent material can be reversible so that the material can be reused and naphthalene and possibly benzene can be recovered after regeneration.

### RELATED APPLICATION(S)

This application claims priority under 35 U.S.C. §119 to European Patent Application No. 11178592.9 filed in Europe on Aug. 24, 2011, the entire content of which is hereby incorporated by reference in its entirety. This application also claims priority under 35 U.S.C. §119 of U.S. Provisional Application No. 61/526,915 filed on Aug. 24, 2011, the entire content of which is hereby incorporated by reference in its entirety.

### TECHNICAL FIELD

The present description is related to the field of hydrocarbon production by gasification of carbonaceous material. For example, disclosed is a method for naphthalene removal from synthesis gas produced by gasification. Also disclosed is the use of a group of adsorbent materials suitable for this application. The process can be utilized as a part of a biomass to liquid (BTL) process.

### BACKGROUND INFORMATION

In the production of hydrocarbon compositions, gasification of carbonaceous material can be utilized as an initial step. Syngas thereby obtained can be converted into hydrocarbons or derivatives thereof, by a synthesis reaction. When gasifying biomass, the raw material can include, and can produce as side reactions, impurities which can be previously uncommon in the field. To optimize the yield and avoid irregularities in the refinement process, employing further process steps can be desirable.

The gasification of carbonaceous material can produce primarily carbon monoxide and hydrogen, thus syngas. Also carbon dioxide, water and different hydrocarbons can be abundant side products in the gasification product. Depending on the source and composition of the carbonaceous raw material, traces and derivatives of other elements can be present as impurities. Some impurities and side products are harmless, for example, nearly inert in the process and may not require any attention. Some side products are easily removed.

Aromatic compounds as gasification side products, for example, can be neither harmless nor easily removed. The simplest aromatic compound, benzene, can be an occupational health problem. It can be desirable to remove benzene from the process as soon as possible to minimize the exposure of plant workers to this carcinogen. Suitable adsorbents for removal of benzene from this kind of gas composition may be employed.

Another aromatic compound of which removal at an early stage can be desirable is naphthalene. It can have a physical tendency to solidify at low temperatures. As a process stream is cooled at a stage, there can be a risk of naphthalene freezing causing clogging and blocking of the process equipment. An efficient commercial adsorbent for naphthalene removal is not known.

When applying other methods, benzene and naphthalene have shown to be problematic components for raw gas purification section as they can have a high vapor pressure even in solid phase. These components, for example, even in small quantities, can flow through cold washes and can cause blocking problems at a wash section and environmental problems at CO₂ purge from the process.

Patent Publication No. GB 316948 discloses removal of nitrogen oxides, cyclic diolefines, and ketones from coke oven gas prior to low temperature cooling. This is effected by treatment with a substance which adsorbs these impurities and also promotes a catalytic action between them. Active carbon and silica gel are suitable adsorbents. They may be impregnated with metals or metal salts. Regeneration of the adsorbents is effected by heating in nitrogen. The coke oven gas is reported to undergo a preliminary removal of higher benzene hydrocarbons, hydrogen sulphide, carbon dioxide, and water. However, the method for said preliminary removal is not disclosed.

U.S. Patent Application Publication No. 2003/0126989 discloses a process for syngas purification, employing a bed of adsorbent(s) comprising an adsorbent based on a zeolite of NaLSX type, with Si/Al ranging from 0.9 to 1.1. It can be considered advantageous compared with beds of adsorbents based on 4A or NaX zeolites, since it can allow longer cycle times and therefore less frequent regenerations. When the syngas to be purified also contains heavy hydrocarbons as impurities, such as butanes, pentanes, etc., it is possible to use the NaLSX-based adsorbent by itself, but in an exemplary embodiment, in the adsorption column, to add to the CO₂-selective NaLSX-based adsorbent one or more adsorbents capable of selectively adsorbing heavy hydrocarbons, such as for example aluminas, silica gels or active carbons, or zeolites. Aromatic hydrocarbons or specific challenges related to their removal are not addressed. U.S. Patent Application Publication No. 2006/0117952 discloses a method applying a NaLSX absorbent, wherein at least 70% of the exchangeable sites are occupied by sodium ions, is combined with another adsorbent, capable of selectively adsorbing each of other impurities except for CO₂ and H₂O, yet another adsorbent capable of selectively adsorbing H₂O, the adsorbents being either intimately mixed or in the form of separate beds in successive layers. The method further involves desorbing carbon dioxide and other impurities adsorbed on the adsorbents by increasing the temperature and/or reducing the pressure, and optionally recycling some of a first purified gas. Here again, the impurities present in the contaminated syngas were discussed generally.

International Publication No. WO 2008/068305 discloses a multistep process for producing a purified synthesis gas stream from a feed of contaminated synthesis gas comprising methanol washes and first and second solid adsorbents. As a first solid adsorbent, sulphur-impregnated activated carbon and/or activated carbon are mentioned removing metals and/or metal carbonyls. The second solid adsorbent comprises one or more metals or oxides of metals or combinations thereof, the metals can be selected from the group of Ag, Sn, Mo, Fe and Zn. A disclosed solid adsorbent is ZnO, because of its good performance in COS content reducing. However, the removal of aromatic hydrocarbons is not discussed in this publication.

Publication No. CA 1041006 (A1) discloses a method for decreasing the naphthalene concentration in debenzolized light oil. Said method comprises pumping the primary light oil condensate and oil bled from a naphthalene scrubber or oil type final cooler to a level above the topmost additional tray in the top portion of the wash oil. No solid adsorbent material is employed for naphthalene removal.

Publication No. CN 101157871 discloses a method combining a rectification technique and freezing crystal technology: first naphthalene in the waste diesel is primarily concentrated by intermission rectification; then naphthalene in the fractioning rich in naphthalene can be separated by the freezing crystal technology; at last kettle fluid with less naphthalene, the lean naphthalene fraction and lean naphthalene oil are mixed so as to regenerate diesel. The content of naphthalene drops down from 3-4 percent to 0.5 percent. However, this technique exploiting the physical characteristics of naphthalene is not applicable to combined benzene and naphthalene removal.

### SUMMARY

It can be desirable for providing an alternative method for removal of aromatic compounds from the process. Further, it can be desirable to remove naphthalene from said process before any step in which the temperature is lowered to a level causing naphthalene to precipitate. It can be desirable to provide for combined benzene and naphthalene removal. When removing benzene and naphthalene from the biomass gasification process, it can be desirable to recover said chemicals to provide benzene and naphthalene of bio-origin. It can be desirable to simplify and increase the effectiveness of the overall process.

According to an exemplary aspect, disclosed is a method involving solid adsorbents comprising benzene provides surprisingly good results for naphthalene removal. This removal can be implemented after gasification of carbonaceous biomass and before the temperature of the gas so produced reaches a temperature below which naphthalene no longer remains fluid. According to an exemplary aspect, a synergistic function of these removal activities can be attained in a way that applying said removal acts in combination and in a set sequence. Exemplary aspects for naphthalene removal or combined benzene and naphthalene removal can be applied to a main stream, but the method can also be applicable to a side stream.

According to an exemplary aspect, the process for benzene and naphthalene removal can be incorporated to any process where naphthalene or both benzene and naphthalene are undesirable. The process can even be applied to a process wherein it is desirable to remove the naphthalene alone. If a beneficial effect of adsorbent function, when pre-exposed to benzene is desired to be exploited, the feed to a first solid adsorbent can be enriched with a sufficient amount of benzene to provide the effect. After said naphthalene removal step, benzene can subsequently be removed, for example, immediately from exhaust of said naphthalene removal step. According to an exemplary aspect, disclosed is a method for naphthalene removal from gas, which can be obtained by gasifying carbon containing raw material to produce a gas comprising carbon monoxide and hydrogen as main components, and at least naphthalene as an impurity. The removal step can be performed by contacting the gas to be purified with the first porous solid adsorbent material comprising benzene. In an exemplary embodiment, the method of naphthalene removal can be applied to production of hydrocarbons or derivatives thereof.

According to an exemplary aspect, disclosed is a method for naphthalene removal from a gas stream, the method comprising: a) gasifying a carbon-containing raw material to produce a gas stream comprising carbon monoxide and hydrogen as main components, and at least naphthalene as an impurity; and b) contacting the gas stream containing naphthalene as an impurity, with a first porous solid adsorbent material comprising benzene.

According to an exemplary aspect, disclosed is a method for producing hydrocarbons or a derivative thereof from a biomass raw material, the method comprising: a) gasifying the biomass raw material in the presence of oxygen to produce a gas comprising carbon monoxide, carbon dioxide, hydrogen and hydrocarbons, wherein the hydrocarbons contain at least naphthalene as an impurity; b) removing naphthalene from the gas obtained in step a) by contacting said gas with a first porous solid adsorbent material comprising benzene; c) adjusting a hydrogen to carbon monoxide ratio of the gas; d) converting in a synthesis reactor at least a part of the carbon monoxide and hydrogen contained in the gas into a product containing a hydrocarbon composition or derivative thereof; and e) recovering the product containing a hydrocarbon composition or derivative thereof.

### BRIEF DESCRIPTION OF THE DRAWINGS

FIG. 1 is a graph of mass spectrum as a function of time giving total benzene and naphthalene responses from an adsorption experiment using helium as a carrier gas, according to an exemplary aspect. The graph shows differences in adsorption and desorption dynamics of benzene and naphthalene, according to an exemplary aspect.

FIG. 2 is a graph of a component analysis from the same adsorption experiment as in FIG. 1 showing concentrations of benzene, toluene and naphthalene as a function of time, according to an exemplary aspect.

FIG. 3 is a graph showing how an adsorbent material (silica gel) performed after multiple regenerations, according to an exemplary aspect. In such exemplary aspect, when using helium as a carrier gas, the recovery was almost perfect, but in regenerations 6 and 7, with carbon dioxide as a carrier gas, the adsorption was considerably disturbed. However, when changing back to helium as a carrier gas, the original adsorption level was almost recovered.

FIG. 4 is a graph of mass spectrum as a function of time giving total benzene and naphthalene responses from an adsorption experiment using helium as a carrier gas, but with a feed poor in benzene, according to an exemplary aspect. The graph shows the dependence of naphthalene adsorption on the presence of benzene, according to an exemplary aspect.

FIG. 5 is a graph illustrating adsorbent area (BET-area and micropore area) and pore size (BET-pore and mesopore) and acidity as a function of total carbon content (benzene+toluene+naphthalene), according to an exemplary aspect.

FIG. 6 is a graph illustrating the correlation between naphthalene adsorbed and the adsorbent area (BET-area and micropore area) pore size (BET-pore and mesopore) and acidity, according to an exemplary aspect. These adsorbent characteristics can be used as criteria for selection for suitable adsorbent materials for naphthalene removal from gases, according to an exemplary aspect.

### DETAILED DESCRIPTION

According to an exemplary aspect, disclosed is a method for naphthalene removal from a gas. Naphthalene removal can include, for example, adsorption of naphthalene present in a gas stream to a solid adsorption material. When applying the method, at least 80%, for example, at least 85%, for example, at least 90% of the naphthalene present in the feed can be removed by the adsorbent material. Herein, naphthalene removal can also be referred to as purification. In an exemplary embodiment, when applying adsorption materials that adsorb benzene, said adsorbed benzene can form a layer on the adsorbent surface dissolving naphthalene, for example, thus facilitating the adsorption, and can help to bind the same to the surface. For example, benzene is believed to act as a mediator facilitating the naphthalene to first transfer from gas phase to benzene containing surface layer, and then to solid adsorbent. Therefore in an exemplary aspect the adsorption material can comprise benzene.

The gas to be purified can originate from gasification of carbonaceous raw material. For example, gasification can produce, as the main stream syngas, a gas comprising carbon monoxide and hydrogen as main components. The gas obtained can contain both benzene and naphthalene as impurities. To remove benzene and naphthalene, the gas obtained from gasification can be contacted with a first porous solid adsorbent material.

An exemplary application is to purify crude syngas after gasification and filtering steps. For example, a catalytic reforming step can also be implemented before the naphthalene removal by adsorption. For example, in such a stream, the benzene content can be between 200 and 2000 ppm and the naphthalene content can be between 10 and 300 ppm. The benzene to naphthalene ratio when contacting the gas with the first solid adsorbent can be at least 1, for example, at least 2, for example, at least 5, to provide dissolution of naphthalene in benzene on an adsorbent surface.

According to an exemplary embodiment, the gas stream from which naphthalene is to be removed can be a stream directly obtained from gasifying the carbon containing raw material. Alternatively, said gas stream from which naphthalene is to be removed can be a stream obtained by first gasifying and then catalytically reforming said gas.

In an exemplary embodiment, the gas can be treated with one or more trivial wash, scrubbing or filtration segments before contacting with the first solid adsorbent, with the proviso that the process conditions are kept such that naphthalene remains in the gas phase. For example, the exemplary conditions can be derived from textbooks, e.g., phase diagrams, in the field. For example, considering the overall BTL (Biomass to Liquid) processes, the temperature of the gas can be kept above 50° C. For example, an exception is physical absorption, for example, a methanol wash conducted at temperatures below 0° C.

In accordance with an exemplary aspect, a method can be applied to remove naphthalene from a side stream, wherein the components of the side stream are originally obtained from gasifying carbon containing raw material, but separated from the main stream. One example is a carbon dioxide exhaust stream. As CO₂ stream can be nearly free from other contaminants, increased adsorbent life time can be achieved by this embodiment.

The gas to be purified can be obtained by gasifying carbon containing raw material. In an exemplary embodiment, said raw material can be of biomass origin, which provides products from renewable resources. When removing benzene and naphthalene from the biomass gasification process, the recovery can provide naphthalene or both benzene and naphthalene of bio-origin, which can be valuable, for example, where non-fossil chemicals or derivatives thereof are appreciated.

Raw material of biomass origin can comprise both benzene and naphthalene as impurities. It can be desirable to remove both benzene and naphthalene together.

In an exemplary embodiment, the gas to be purified can already comprise the benzene which is used to adsorb to the adsorbent surface. In an experiment, for example, it can be shown that from such gas composition, benzene can be adsorbed first and after that, naphthalene. However, in an exemplary embodiment, the subsequent and/or further removal of benzene is exemplary. According to an exemplary embodiment, the porous solid adsorbent material can adsorb benzene reversibly. For example, this can apply to both a first adsorbent material as well as a second adsorbent material.

In an exemplary embodiment, applying a method does not require both benzene and naphthalene to be inherently present in the same gas composition. In cases where gas to be purified does not contain any benzene or enough benzene, benzene can be added prior to naphthalene removal to ensure efficient adsorption of the naphthalene. For example, one option is to add benzene to the gas stream to be purified upstream to the adsorbent. This can be accomplished by adding an inlet for benzene before an adsorption unit, for example, immediately before the adsorption.

For example, another option for providing the first solid adsorbent material comprising benzene, is to pre-saturate the adsorption material with benzene. This can be combined with the addition of benzene to the gas stream as described above. This can be designed so that the adsorbent unit comprising said pretreated adsorption material is installed as the first adsorption material to the gas stream intended for naphthalene removal.

For example, the benzene either inherently present in the gas to be purified, or added to it or used for pre-saturation of the adsorbent material, can be removed downstream from the naphthalene adsorption unit. The benzene can be removed by any suitable means. Adsorbent materials applicable for benzene removal only can be employed. Circulation of benzene from exhaust to feed can be employed. For example, this can be performed using second porous solid adsorbent material suitable for benzene adsorption. It can include the same material as the first porous solid adsorbent material. This can offer an exemplary advantage of interchanging said adsorbents providing material pre-saturated with benzene for naphthalene removal. Alternatively, the first and second porous solid adsorbent materials can partially comprise the same adsorbent materials.

Using same or similar materials can provide an exemplary benefit in that said materials can be interchangeable and material already having benzene adsorbed thereon can be readily applicable for naphthalene removal. In an exemplary embodiment wherein adsorbent is pre-saturated with benzene, this can be arranged by flexible process design.

In an exemplary embodiment, the adsorption materials may also be different from one another. In the case where different adsorbent materials are used for naphthalene and benzene removal, the experiments conducted can provide guidance for selecting the adsorbent. For example, adsorbent materials for naphthalene removal can include non-acidic mesoporous materials which, for example, are not microporous. Exemplary materials may be selected from aluminosilicates and silica gel. Exemplary adsorption materials for benzene removal can be microporous nonacidic adsorbents such as, for example, zeolites and activated carbon.

The adsorbent materials applicable to an exemplary process can comprise chemically variable range of solids. Materials can have average pore size of 2-50 nm. For example, adsorbent materials suitable for naphthalene removal can be used for combined naphthalene and benzene removal. For example, the surface area of the micropores of the solid adsorbent material used can be less than 300 m²/g. Based on experiments conducted, the first solid adsorbent material can be selected from activated carbon, silica gel, molecular sieves, zeolites and aluminosilicates. As shown in the experiments, the characteristics and strengths of adsorbent materials can vary and can provide solutions applicable to different conditions and aims. For example, silica gel as naphthalene adsorbent showed excellent regeneration characteristics, but at the same time, can be vulnerable to high CO₂ content in the feed.

One group of suitable adsorbents can contain silicon, oxygen and optionally aluminum. They can have pore sizes above 2 nm. These adsorbent can be easily regenerated repeatedly and can be chemically very stable. In an exemplary embodiment, they do not contain any poisons or environmentally unacceptable components. The adsorbent structure can fall within certain physical characteristics ranges.

Any suitable method for measuring the average pore size in adsorbent materials cam be employed. Pore volume, pore diameter and micropore area can be measured by N₂-physisorption (N₂ adsorption-desorption). Pore volume can be obtained by N₂-physisortpion utilizing BET equation (Brunauer-Emmett-Teller, ASTM D 4641). Average pore diameter can be calculated from N₂-physisorption utilizing BET or BJH equation (Barrett-Joyner-Halenda ASTM D 4641). Micropore area can be defined by N₂-physisorption utilizing t-Plot method.

The adsorption materials can be packed in a reactor acting as an adsorption unit. In the process from gasification to further processing of the syngas purified from naphthalene or both naphthalene and benzene, the adsorption can take place in at least one adsorption unit. For example, for a gas comprising both naphthalene and benzene, at least two adsorption units can be applied, the first of which comprises first porous solid adsorbent material comprising benzene for removing naphthalene from the gas to be purified; and the second comprises second porous solid adsorbent material adsorbing mainly benzene. The presence of benzene can increase naphthalene adsorption, so it can be desirable to place a naphthalene adsorption unit upstream with respect to a benzene adsorbent.

For example, first and second units can comprise the same adsorbent material enabling the process equipment to be arranged in a way such that an adsorbent unit previously acting as the second porous solid adsorbent material adsorbing benzene can be installed as the first adsorbent unit for the naphthalene adsorption. Any other suitable arrangement involving a larger number of units following the same logic can be employed, for example, connecting several units in series or by other arrangement, in accordance with an exemplary aspect.

In an exemplary embodiment, for some adsorbent materials, improved performance can be achieved if the gas entering the adsorbent unit is free from distracting compounds compromising their capacity. For silica gel as adsorbent material such distracting compounds can include, for example, water (H₂O), carbon dioxide (CO₂) and hydrogen sulfide (H₂S). One or more of these compounds can be removed from the gas applying a wash step upstream with respect to the adsorbent unit.

For example, the first solid adsorption material can adsorb naphthalene in a reversible way. For example, it is possible to regenerate the adsorption material for reuse. When applying the present method in a way that also includes benzene removal, for example, the second porous solid adsorbent material can adsorb benzene reversibly. An exemplary advantage with reversible adsorption is the cost efficiency obtained by reuse of the solid adsorbent material.

Depending on the adsorbent and the level of purity desired, for example, three procedures for regeneration can be employed. The most simple and cheapest method for regeneration can be flash regeneration, wherein the adsorbent pressure is decreased. By employing vacuum in the last step, the aromatic compound concentration in the adsorbent can further be lowered.

For example, in adsorption technology, after saturation of the adsorbent with one or more components, any such component may be desorbed by reducing the partial pressure of that component in the atmosphere surrounding the adsorbent. As an example of such idea, the use of a pressure differential cycle employing molecular sieve adsorbents is disclosed in G. J. Griesmer et al., Latest Advances in Selective Adsorption, Petroleum Refiner, Vol. 39, No. 6, pages 125-132 (June 1960).

For example, in pressure differential cycles, the pressure reduction at the beginning of the desorption step can be carried out in a variety of ways. For example, when the adsorption process has been carried out for the purpose of obtaining a pure effluent, and when adsorption has been conducted at super-atmospheric pressure, the saturated adsorbent may be merely vented to the atmosphere to desorb any unwanted adsorbed component. If it is desired to retain the adsorbed material, however, again in a super-atmospheric operation, the venting may be into a closed vessel, thereby permitting the recovery of the adsorbed material.

For example, when higher purity is desired, the regeneration can be performed by stripping the adsorbent with an inert gas. In stripping, the adsorbent pressure can be lowered and thereafter the partial pressures of the gases to be removed can be decreased by feeding inert gas to the reactor. A negative side of this regeneration system can be the dilution of aromatic compound stream with the inert gas used.

For example, in some cases, hot regeneration can be desirable. This can provide a very high degree of purity for the gas to be purified and additionally aromatic compound concentration in the effluent gases. For example, said regeneration of the solid adsorbent material can be performed by heating at a temperature of less or equal to 250° C. and ambient pressure. However, any pressure lower than that of the adsorption is possible, even vacuum.

As an exemplary embodiment of an overall process, the naphthalene removal can be applied in a production process for hydrocarbons or derivatives thereof from biomass raw material. The method can comprise the steps of gasifying the biomass raw material in the presence of oxygen to produce a gas comprising carbon monoxide, carbon dioxide, hydrogen and hydrocarbons; removing naphthalene from the gas obtained in the previous step by contacting the gas with a first porous solid adsorbent material comprising benzene; adjusting the hydrogen to carbon monoxide ratio; converting in a synthesis reactor at least a significant part of the carbon monoxide and hydrogen contained in the gas into a product selected from hydrocarbon composition and derivatives thereof; and recovering the product hydrocarbon or derivative thereof.

This method can produce hydrocarbons or derivatives thereof of biomass origin. The composition of the product gas can depend on the biomass raw material resource. For example, wood, bark, cereals, straw, bagasse, etc. are all possible raw materials for biofuel production. For ethical reasons, non-food raw materials can be exemplary.

For example, gasifying the biomass raw material for producing a syngas can take place in the presence of oxygen. For example, gasifying the raw material in the presence of oxygen can be performed at a temperature of at least 1000° C. At these conditions, biomass, such as lignocellulosic materials, can produce a gas containing carbon monoxide and hydrogen, thus the components of syngas, as well as carbon dioxide and water gas. It can contain some hydrocarbons and impurities, such as sulfur and nitrogen derivatives and trace metals and derivatives thereof.

Optionally the gas obtained from gasification can be subjected to catalytic refining. For example, the method can further comprise at least one catalytic reforming step, for example, before naphthalene removal.

For example, in an exemplary embodiment, the exhaust from gasification or from catalytic refining is not an optimal feed for synthesis reactors. For example, the hydrogen to carbon monoxide ratio can be low, for example, from 0.5 to 1.0 after gasification and optional reformation, and therefore it can be desirable to increase the ratio. Adjusting the hydrogen to carbon monoxide ratio can aim at raising said ratio to a value of at least 1.5, for example, about 2. Any suitable approach to change this ratio can be employed such as, for example, Water Gas Shift either for sour gas or purified gas.

An example of the synthesis reaction producing hydrocarbons is Fisher-Tropsch-synthesis (FT). For example, in the FT reactor, at least a part of the carbon monoxide and hydrogen contained in the gas can be converted into a hydrocarbon composition comprising C₄-C₉₀ hydrocarbons, in other words, hydrocarbons having carbon numbers in the range of from 4 (included) to 90 (included). The products obtained in the Fischer-Tropsch reaction, for example, said C₄-C₉₀ hydrocarbons, can include distillates and hydroconverted products, for example, fuels such as naphtha, kero and diesel, base oils and n-paraffins, light detergent feedstocks and wax.

The Fischer-Tropsch synthesis can be carried out at a temperature in the range from 125 to 350° C., for example, from 200 to 260° C. The pressure can range from 5 to 150 bar, for example, from 5 to 80 bar absolute. In the Fischer-Tropsch synthesis, for example, more than 65 wt % of C₄-C₉₀ hydrocarbons, for example, more than 85 wt % C₄-C₉₀ hydrocarbons can be formed. Depending on the catalyst and the conversion conditions, the amount of heavy wax C₂₀-C₉₀ hydrocarbons may be up to 60 wt %, for example, up to 70 wt %, for example, up to 85 wt %.

For example, when applying to this method at least one step of benzene removal, benzene of bio-origin can be separated and further purified for release to chemical market or alternatively for reuse in this process. Removal of benzene can also contribute to occupational safety. Incorporating at least one wash step can increase the product purity and can remove distracting compounds, for example, catalysts in process steps following said washes.

### EXAMPLES

1 Adsorption Experiments

1.1 Experimental Equipment

Adsorption experiments were conducted using equipment including three tube reactors, of which the first in upstream was a flashback arrestor, second the bubbler of naphthalene/benzene mixture and the third housed the adsorbent of interest. The bypass routes and pressurizing aids used for the operation of equipment were arranged by piping, closings and three-way valves. All the surfaces of the equipment being in contact with hydrocarbons were thermosealed to avoid desublimation of naphthalene. The pressure of the experimental equipment was settled with a string-adjusted relief valve and the pressure could be monitored by local pressure gauges. Carrier and dilution gas were brought to the equipment, the flow of which was adjusted using mass flow instruments (Bronkhorst).

The composition of the outlet gas from the adsorbent was measured with a mass selective detector and two FIDs, to which the gas stream was lead using a valve system. The product gas composition was monitored with a mass-selective detector, which gave the gas component distribution. Similarly, the first FID (front detector) was used for monitoring the overall response. The second FID (hind detector) was used for measuring the product gas composition as its components by samples taken from the gas stream at regular intervals. The quantification of the components observed in all three detectors and the overall response were based on external calibration, wherein the calibration response was obtained from a commercial gas mixture (AGA).

### Conduct of the Experiments

The adsorbent was loaded in a tube reactor with an outlet (1.00 mm) for gas flow. A screw held the adsorption material in place during the adsorption experiment. The adsorbent was centralized by the length of the reactor and layers of glass wool and glass powder were loaded upstream and downstream of the adsorbent to stabilize the flow.

The slowest steps of the adsorption experiment were the increase of the pressure and stabilizing the concentrations before initializing the adsorption phase. Prior to the adsorption phase, the composition of the gas fed to adsorbent was analyzed by its components and as total response. During the test run, samples for the separation column component analysis were taken regularly. Saturation of the adsorbent material was recognized by a sudden increase of the overall response. The adsorption phase was continued until the overall response returned to the original level, after which the feed of hydrocarbons was ceased by bypassing the bubbler of naphthalene/benzene mixture. Then, the adsorbent was rinsed with the carrier gas to remove traces of hydrocarbons before regeneration.

Regeneration was initialized by dropping the pressure of the system to the ambient, and then raising the temperature to 250° C. The temperature was maintained in the set value and the experiment was continued until the amplitude of the overall response was lowered as close to the base line as possible. The adsorption capacity as a percentage in relation to the mass of an adsorption material was calculated from the overall response of the regeneration. The division of the hydrocarbons adsorbed was derived from the responses of the different components on the mass-selective detector.

1.2 Adsorption Materials

Adsorption experiments were conducted testing commercially available materials as well as exemplary adsorbents (activated carbon, silica gel and molecular sieves). Some tailored mesoporous materials were included in testing as well (Table 1). Physical characteristics for said materials were measured including BET-area [m²/g], surface area of the micropores (microarea) [m²/g], pore volume [cm³/g ], mesopore diameter [Å], BET-pore diameter [Å], Zeolite pore size [Å] and acidity.

TABLE 1

| | | Micropore | Pore | Mesopore | BET pore | |
|---|---|---|---|---|---|---|
| | BET-area | area | volume | diameter | diameter | Acidity |
| Adsorbent | (m²/g) | (m²/g) | (cm³/g) | (Å) | (Å) | μmol/g) |
| Commercial adsorbent 1 | 615 | 587 | 0.369 | 193 | 24 | low |
| Commercial adsorbent 2 | 465 | 364 | 0.358 | 77 | 31 | low |
| Commercial adsorbent 3 | 421 | 269 | 0.498 | 87 | 47 | 25 |
| Commercial adsorbent 4 | 301 | 31 | 0.900 | 40 | 119 | 534 |
| Tailored mesoporous 1 | 1130 | 0 | 0.990 | 26 | 35 | 62 |
| Tailored mesoporous 2 | 832 | 25 | 0.770 | 26 | 37 | 190 |
| Tailored mesoporous 3 | 650 | 123 | 1.250 | 60 | 80 | 25 |
| Tailored mesoporous 4 | 624 | 16 | 0.510 | 24 | 33 | 25 |
| Aluminiumsilicate | 465 | 0 | 0.760 | 38 | 65 | 353 |
| Molecular Sieve13X | 556 | 528 | 0.330 | 40 | 24 | low |
| Activated carbon | 1360 | 1264 | 0.695 | 31 | 21 | low |
| Silica gel | 722 | 139 | 0.427 | 35 | 24 | low |

### Experimental Conditions

The pressure in the experiment equipment was raised to 8 bar and the temperature on the benzene/naphthalene was kept at ambient, 24° C. The adsorbent temperature was set to 40° C. The carrier gas stream through the hydrocarbon mixture and adsorbent was 2.0 dm³/h. The benzene concentration in the experiment conditions was 1.2-1.8 vol-% and the naphthalene concentration was 0.001-0.003 vol-%. The adsorbent was loaded (40-200 mg) to the reactor to form a layer having a height of at least 20 mm in the reactor tube having an inner diameter of 4.0 mm. In the experiments conducted, use of the same average particle size of 75-150 μm as far as possible was ensured by grinding or sieving from other particle distributions. This was not possible for all the materials used as some of them were only available as very fine powders or 20-40 mesh particles (e.g., activated carbon).

In the preliminary experiments, helium was used as the carrier gas. The experiments were thereafter repeated using calibrating gas mimicking synthesis gas and sulfur containing synthesis gas obtained from gasification of biomass raw material in a gasification pilot-plant. Composition of the carrier gases are compiled in Table 2.

TABLE 2

| | Carrier gas | | | | | |
|---|---|---|---|---|---|---|
| | CO | CO₂ | H₂ | CH₄ | N₂ | H₂S |
| | vol-% | vol-% | vol-% | vol-% | vol-% | ppm |
| Calibrating gas | 15.0 | 14.9 | 14.8 | 3.1 | 52.2 | — |
| Synthesis gas | 20.7 | 29.1 | 34.4 | 3.0 | 11.7 | 156 |

Small amounts of water (0.02 vol-% H₂O) and ammonia NH₃, as well as an ethylene peak appearing between carbon dioxide and water in the separation column were observed in the synthesis gas.

1.3 The Different Stages of the Adsorption Measurements

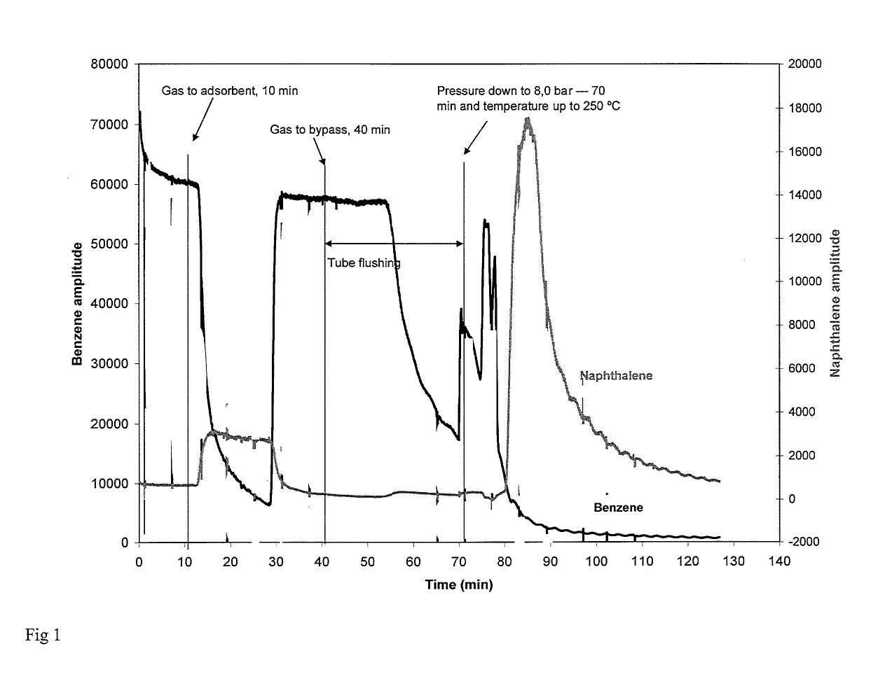

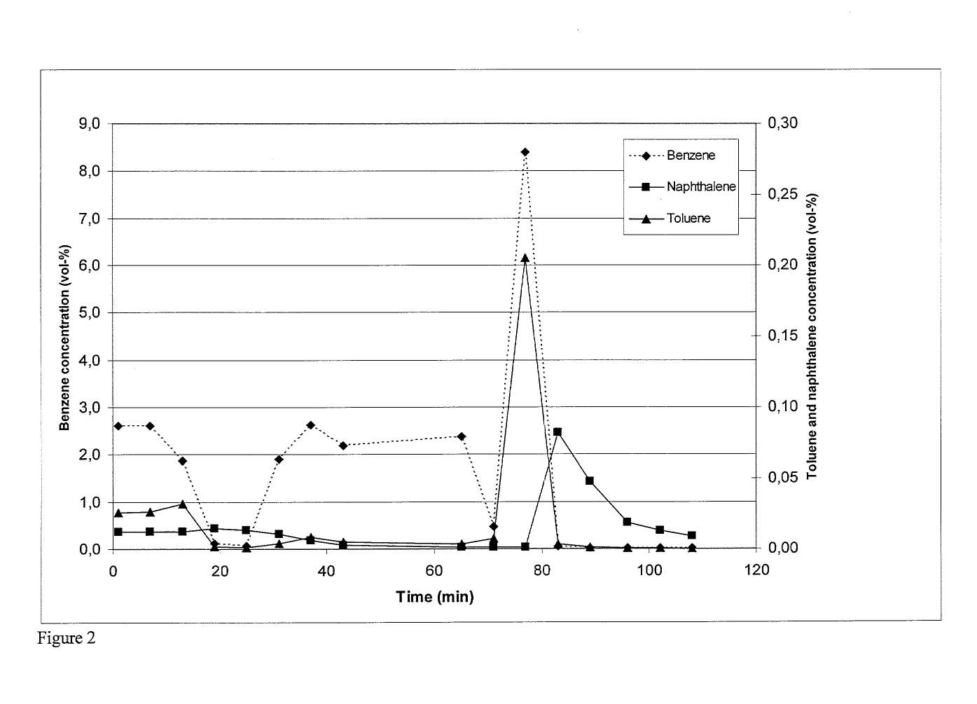

All the materials studied followed the same general behavior as a function of time. Therefore a detailed disclosure of one experiment can be considered representative of all the experiments. The most relevant differences can be observed in speed of impregnation and saturation with hydrocarbons and the readiness for regeneration between the materials. Responses as a function of time are given in FIGS. 1-2 using as a model material. The first of these figures, FIG. 1, shows the benzene and naphthalene responses as a function of time. The second figure, FIG. 2, presents a component analysis showing concentrations of benzene, toluene and naphthalene as a function of time.

All the measurements (except the one reflecting low benzene concentration) showed an increase in the concentration of naphthalene simultaneously to the initiation of the benzene adsorption. For example, the withdrawal of benzene from the gas phase may cause a change in partial pressures and consequently allows a larger amount of naphthalene to vaporize to the carrier gas.

This can be seen in FIG. 1, wherein the response of naphthalene increased at point 23-25 min and a small delay in naphthalene adsorption could be seen in respect to the benzene adsorption. As the adsorbent was saturated with the hydrocarbons, benzene bypassed the adsorbent without being adsorbed, which was showed as the return of the benzene response to the original level. On the contrary, the adsorption of the naphthalene continued and the adsorbent was not saturated with naphthalene at any point of the experiment.

The continuous naphthalene adsorption could also be seen in the component analyses in FIG. 2.

The differences in the behavior of benzene and naphthalene were also evident during the regeneration of the adsorbent material. Benzene left the adsorbent material in a lower temperature, whereas a temperature nearly as high as 250° C. was employed to desorb naphthalene from the adsorbent. The regeneration was also affected by the amount adsorbed hydrocarbons. It was noted that a material which had adsorbed a considerable amount of naphthalene exhibited a longer time to return the signal to the base line.

2 Results

2.1 Measurements with Helium as Carrier Gas

In these experiments, experimental conditions were set up to compare the performance of different adsorbent materials, compare adsorption of different hydrocarbons, and study the dynamics of the naphthalene adsorption.

The adsorbents were aligned by the performance in the naphthalene removal. The values were collected from the component analyses at the initiation of the experiment and at the end of the adsorbing phase, just before the adsorbent mass was saturated with benzene. In such example, activated carbon showed to be the best adsorbent for all hydrocarbons measured. The concentration of benzene in the carrier gas decreased from a value of 15570 ppm to as low as 140 ppm, the toluene concentration from 103 ppm to 0.6 ppm and naphthalene from 34 ppm to 1.4 ppm. Other results used for finding correlations between the adsorbent characteristics and performance in experiments are not shown here.

The performance of all adsorbent materials with respect to the removal of molecules smaller than naphthalene was comparable. Nevertheless, the adsorption of naphthalene showed more obvious differences between adsorbent material performances.

Considering the mass of the adsorbent material used for the experiment and calculating a change in the concentration of hydrocarbons in relation to the mass of adsorbent material (1000 mg) in the experiment, another alignment was achieved. This alignment is not shown, but used for correlation creation. The accuracy of these calculations has some weaknesses as the result obtained for some of the samples was obtained from the range of rapid change and on the other hand, none of the experiments was driven to the point of saturation with naphthalene. Based on the calculated results, Commercial absorbent 3 showed best performance under conditions of the experiment.

The exemplary adsorbents were also compared and aligned in the order of best adsorption of total hydrocarbons (benzene+toluene+naphthalene). In such example, activated carbon exhibited the best adsorbent characteristics for the adsorption of hydrocarbons, for example, benzene. The amount of hydrocarbons adsorbed is calculated as the response of hydrocarbons released from the adsorbent material when subjected to regeneration conditions, at the temperature of 250° C. The calculated naphthalene amounts do not reflect the case where the adsorbent material binds the hydrocarbons to its structure so that regeneration is not complete.

2.2 Measurements with Sulfur Free Model Gas as Carrier Gas

Sulfur free model gas as carrier gas was studied using as Commercial adsorbent 3, because it showed best results for both benzene and naphthalene and relative good results for total hydrocarbon adsorbing in an example. Experimentally, it was found that when using the model gas, the percentage adsorbed was 10.3 wt-%, which was slightly higher than that measured with helium (average from 4 measurements). The higher value was due to the presence in the model gas of hydrogen, which is a flame gas for FID bringing about an increase in the detector signal. Calibration response was obtained with a hydrogen free hydrocarbon mixture. However, large molecules in the model gas shadowed the compounds having a response area overlapping that of the ionization area, thus rendering it difficult or impossible to measure the component distribution due to erroneous response relations. Even though the component distribution was not obtained, the results of this experiment show that the components of the model gas do not disturb the operation of the adsorbent. Thus, based on this experiment, naphthalene or naphthalene and benzene removal from synthesis gas using adsorption pattern as described here can be applied to after-gasification process.

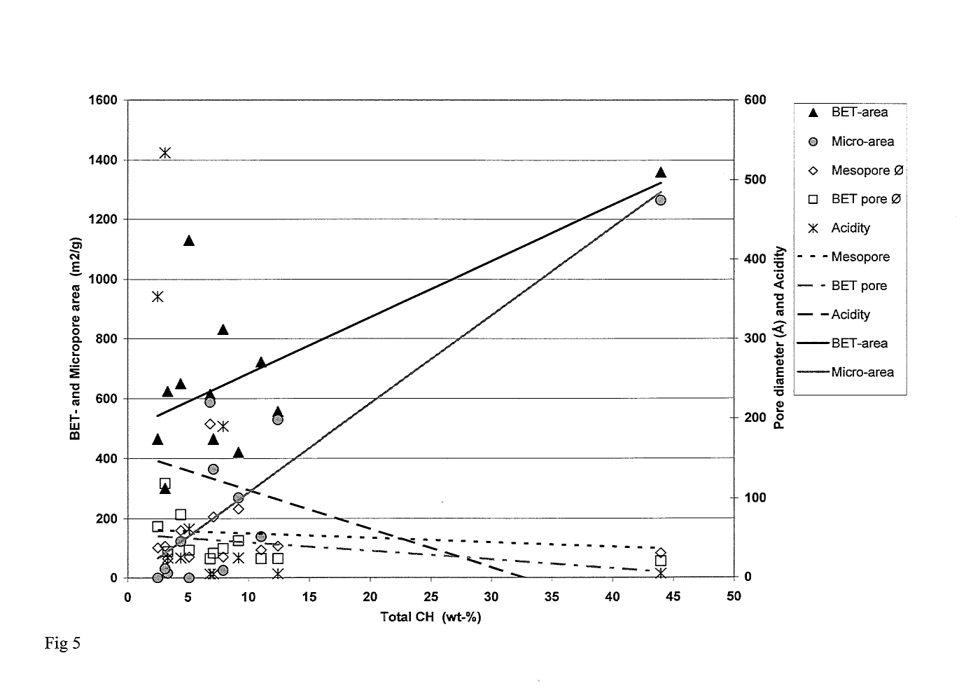

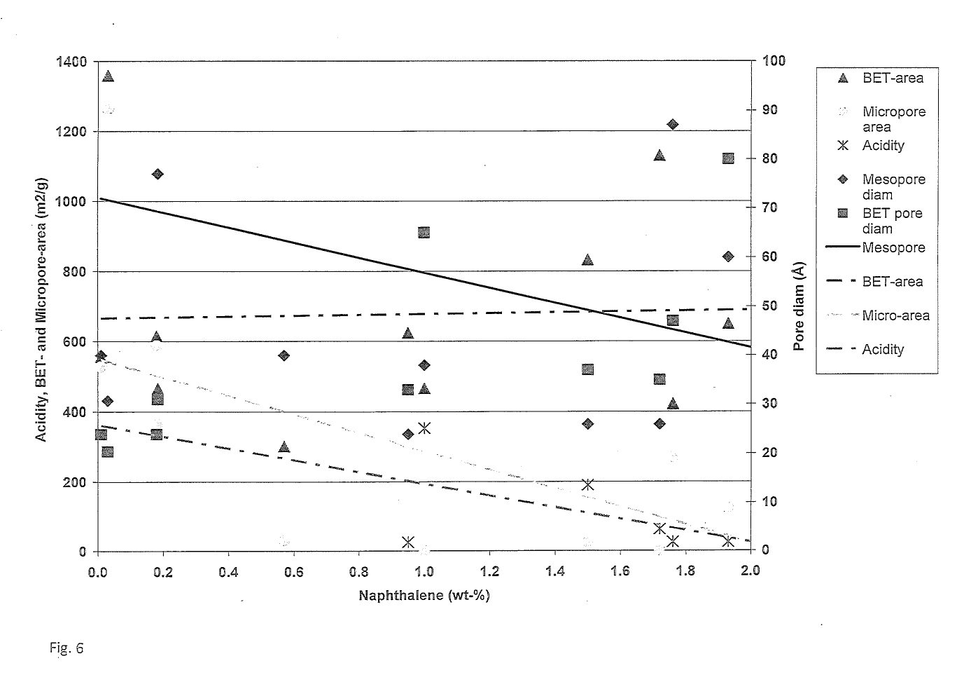

In the examples, the most efficient and easily regenerated adsorbent for removing benzene and naphthalene from synthesis gas is silica gel. Presence of benzene on surface of adsorbent is desirable for naphthalene adsorption. High concentration of carbon dioxide decreases adsorption capacity and even low concentration of water prevents totally removing of hydrocarbon contaminants from gas. According to correlation of measured physical values and adsorption capacity, a good adsorbent for removing benzene and toluene is an adsorbent material with a large surface area (BET and surface area of the micropores) and low acidity (FIG. 5). An efficient adsorbent for naphthalene is a material with large pore volume, low micro porosity and low acidity, for example, less than 30 (FIG. 6).

2.3 Measurements with Sulfur-Containing Synthesis Gas as Carrier Gas

The effects of sulfur-containing synthesis gas as the carrier gas for adsorption were studied using silica gel as adsorbent material. Synthesis gas was obtained from gasification of biomass in a pilot plant. Adsorption capacity was lowered from 8.3 wt-% obtained with helium as carrier to 6.2 wt-% with sulfur containing synthesis gas as a carrier. Regeneration showed release of some H₂S, so silica gel adsorbs both hydrocarbon and sulfur compounds. The component distribution was not reliable, as large molecules in the synthesis gas shadowed the compounds having a response area overlapping that of the ionization area. As a conclusion, sulfur compounds, for example, hydrogen sulfide seem to disrupt the adsorption of naphthalene or naphthalene and benzene from gas stream. Therefore, removal of sulfur derivatives seems worthwhile before naphthalene or naphthalene and benzene removal when applying silica gel as adsorbent material in the process. For example, removal of sulphur derivatives, thus acid gases is conducted using methods of chemical absorption, whereby the reaction conditions can be kept such that naphthalene remains in the gas phase.

2.4 Measurements with Carbon Dioxide as Carrier Gas

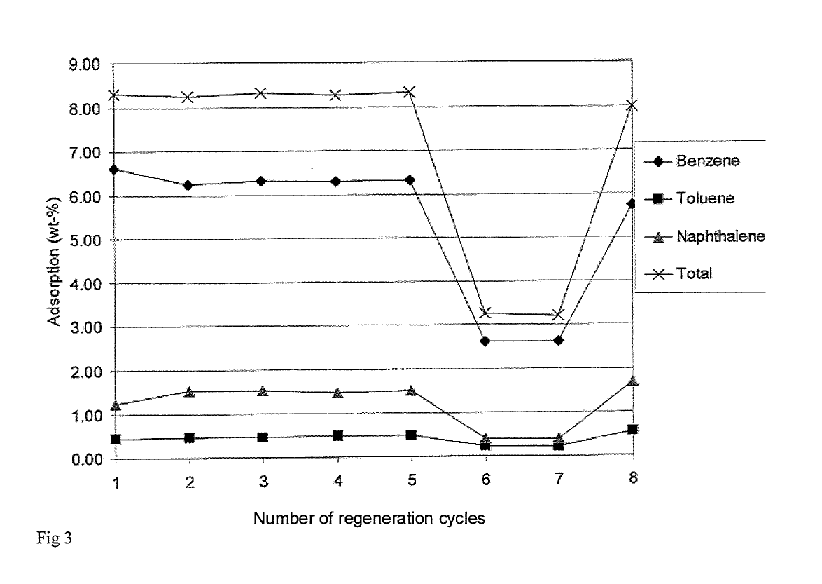

Carbon dioxide (100%) as the carrier gas was studied using silica gel as the adsorbent material. The adsorption decreased for substantially all hydrocarbons (benzene, toluene and naphthalene). In FIG. 3, all the other experiments were conducted using helium as carrier, but 6 and 7, wherein carbon dioxide was used. When regenerating the adsorbent in the carbon dioxide stream, only about one third of the naphthalene bound to adsorbent material was released and removed. From this, it can be concluded that when using silica gel for removing hydrocarbons from CO₂, the heavy molecules probably accumulate on the adsorbent material if the regeneration takes place at a temperature lower than 250° C. When regenerating silica gel in helium flow, the adsorbent capacity was almost completely recovered. This conclusion is applicable to silica gel as adsorption material. Other adsorption materials could be more tolerable to carbon dioxide and even suitable for regeneration with CO₂.

By regenerating the silica gel in helium stream, the adsorption capacity was almost completely recovered (regeneration 8 in FIG. 3).

2.5 The Effect of Water on Adsorption

When the carrier gas fed to adsorbent (both silica gel and Commercial absorbent 3 tested) contained a small amount of water (0.2 vol-%, 8 bar, 50° C.) in addition to the hydrocarbons (benzene+toluene+naphthalene), the adsorbent lost all its purification capacity. During the adsorption phase, benzene was adsorbed on the adsorbent material, but the water and naphthalene concentrations in the carrier gas were increased. The total amount of hydrocarbons adsorbed on the adsorbent material remained substantially the same as in helium flow, but the distribution was changed drastically. The amount of benzene retained on both the adsorbents fell to only one third of that in helium flow and the amount of the naphthalene multiplied to three-fold, but even though, the gas was not purified from hydrocarbons.

2.6 Naphthalene Adsorption from Gas Poor in Benzene

The experiments showed that the adsorption of benzene and naphthalene did not occur simultaneously. This can be due to solvent effect by the lighter component, benzene. To access this effect, the naphthalene adsorption was measured in conditions containing light hydrocarbons benzene and toluene as little as possible. Both aromatic solvent components were present in the carrier gas during the experiment. They originate either from naphthalene as impurity (grade >99, Merck) or they were eluted from the experiment equipment. The measurements were conducted for two different adsorbent materials.

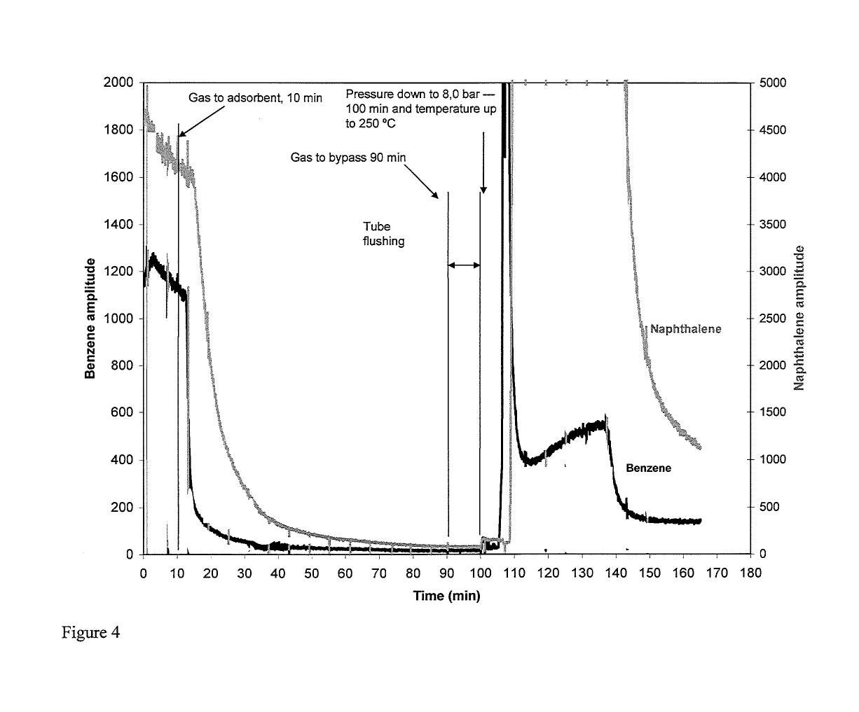

The mass spectrum responses showed the naphthalene adsorption to be slower than that of benzene (FIG. 4). Both experiments confer the conclusion that the benzene adsorption occurring prior to naphthalene adsorption enhances the naphthalene binding (dissolution) to the adsorbent. For example, benzene can form a liquid film on the adsorbent surface facilitating the naphthalene dissolution and thus the dissolving effect enhances in collaboration with the adsorption forces the binding of the naphthalene into the adsorbent material. The amount of the naphthalene bound was thus increased in correlation to the length of the adsorption phase. When driven long enough, the adsorbent total hydrocarbon amount (benzene+naphthalene) approaches theoretical maximum. For example, naphthalene can be capable of substituting benzene in the adsorption sites on the surface of the material. For example, the hydrocarbon adsorption can be about three different equilibriums, the equilibrium of the hydrocarbon partial pressures, dissolution balance and adsorption/desorption competition on the adsorption sites.

For naphthalene removal, it can be desirable to use a solid adsorbent material comprising benzene. Benzene contributes to naphthalene transfer and dissolution from gas phase to the adsorbent material and adsorption sites.

The dissimilarity between naphthalene and benzene was evident during regeneration, as the benzene was released from the adsorbent at a lower temperature and naphthalene only when the temperature approaches 250° C.

3 Regeneration of the Adsorption Materials

The carbon content retained in the adsorption materials after the last regeneration step (He, 250° C.) was measured with TPD/TPO-MS-analyzer from a small sample of the adsorbent material of interest. The parameters compared were carbon content, the maximum temperature in the response profile, the character of the carbon (charcoal, soft/hard charcoal), the residual percentage and total residue (%). The results are not listed here, but used for correlation finding.

Based on the TPD and TPO analyses, silica gel contained less carbon after regeneration (2.7%) and was thus easiest to regenerate in such example. On the other hand, samples of both activated carbon and molecular sieve were the most difficult to regenerate in such example, and a temperature of almost 500° C. was employed for complete regeneration.

The carbon contained in the adsorption materials in most cases suffered a coking reaction as their portion of “hard coke” revealed in the TPO-analyses was considerable large (94.6%, 97.4%, 73.9% and 94.5% respectively). In the coking reaction, the aromatic compounds have formed in oligomeration processes forming even larger multi-cyclic hydrocarbons containing only a small amount of hydrogen, thus coke.

The readiness for regeneration in modest and reasonable conditions can be relevant to industrial feasibility of these adsorption materials. Therefore, materials showing good regeneration results can be suitable as adsorption materials for syngas purification.

It will be appreciated by those skilled in the art that the present invention can be embodied in other specific forms without departing from the spirit or essential characteristics thereof. The presently disclosed embodiments are therefore considered in all respects to be illustrative and not restricted. The scope of the invention is indicated by the appended claims rather than the foregoing description and all changes that come within the meaning and range and equivalence thereof are intended to be embraced therein.

What is claimed is:

1. A method for naphthalene removal from a gas stream, the method comprising:
a) gasifying a carbon-containing raw material to produce a gas stream comprising carbon monoxide and hydrogen as main components, and at least naphthalene as an impurity; and
b) contacting the gas stream containing naphthalene as an impurity, with a first porous solid adsorbent material comprising benzene.

2. The method according to claim 1, wherein at least 80% of the naphthalene present in the gas stream containing naphthalene as an impurity, is removed by the adsorbent material.

3. The method according to claim 1, wherein the gas stream contains benzene as an impurity, and wherein benzene is at least partially removed from the gas stream during step b).

4. The method according to claim 3, further comprising:
c) at least partially removing benzene from the gas obtained from step b) by contacting the gas obtained from step b) with a second porous solid adsorbent material.

5. The method according to claim 1, wherein a surface area of micropores of the first porous solid adsorbent material is less than 300 m²/g.

6. The method according to claim 1, wherein the first porous solid adsorbent material has an acidity of less than 30.

7. The method according to claim 1, wherein the first porous solid adsorbent material is selected from the group consisting of an aluminosilicate, a silicagel and a combination thereof.

8. The method according to claim 4, wherein the second porous solid adsorbent material is selected from the group consisting of a zeolite, an activated carbon and a combination thereof.

9. The method according to claim 1, wherein the first porous solid adsorbent material adsorbs naphthalene reversibly.

10. The method according to claim 4, wherein the second porous solid adsorbent material adsorbs benzene reversibly.

11. The method according to claim 1, further comprising:
adding benzene to the gas stream containing naphthalene as an impurity at a location that is upstream with respect to the first porous solid adsorbent material, and/or
pre-saturating the first porous solid adsorbent material with benzene prior to contacting the first porous solid adsorbent material with the gas stream containing naphthalene as an impurity.

12. The method according to claim 1, further comprising regenerating the first porous solid adsorbent material.

13. The method according to claim 1, further comprising regenerating the first porous solid adsorbent material by heating at a temperature of less than or equal to 250° C.

14. The method according to claim 1, wherein the carbon-containing raw material is a biomass.

15. A method for producing hydrocarbons or a derivative thereof from a biomass raw material, the method comprising:
a) gasifying the biomass raw material in the presence of oxygen to produce a gas comprising carbon monoxide, carbon dioxide, hydrogen and hydrocarbons, wherein the hydrocarbons contain at least naphthalene as an impurity;
b) removing naphthalene from the gas obtained in step a) by contacting said gas with a first porous solid adsorbent material comprising benzene;
c) adjusting a hydrogen to carbon monoxide ratio of the gas;
d) converting in a synthesis reactor at least a part of the carbon monoxide and hydrogen contained in the gas into a product containing a hydrocarbon composition or derivative thereof; and
e) recovering the product containing a hydrocarbon composition or derivative thereof.

16. The method according to claim 15, further comprising at least one step of removing benzene from the gas obtained from the step b).

17. The method according to claim 15, further comprising at least one washing step.

18. The method according to claim 15, further comprising at least one catalytic reforming step.

19. The method according to claim 1, wherein at least 85% of the naphthalene present in the gas stream containing naphthalene as an impurity, is removed by the adsorbent material.

20. The method according to claim 1, wherein at least 90% of the naphthalene present in the gas stream containing naphthalene as an impurity, is removed by the adsorbent material.

21. The method according to claim 4, wherein the first porous solid adsorbent material adsorbs naphthalene reversibly.
